SEQUENCE LISTING (34 sequences)

SEQ ID NO: 1 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 2 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 3 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatctctggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 4 - protein, 382 aa, Unknown
  Obtained from environmental sample
MKSLFALSLFAGLSVAQNAAWAQCGGNGWTGSKTCVSGYKCTVVNEWYSQCISGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 5 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaaaagactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 6 - protein, 382 aa, Unknown
  Obtained from environmental sample
MKSLFALSLFAGLSVAQNAAWAQCGGNGWTGSKTCVSGYKCTVVNEWYSQCIPGTAEEKTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 7 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacatactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 8 - protein, 382 aa, Unknown
  Obtained from environmental sample
MKSLFALSLFAGLSVAQNAAWAQCGGNGWTGSKTCVSGYKCTVVNEWYSQCIPGTAEEHTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 9 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactcctaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 10 - protein, 382 aa, Unknown
  Obtained from environmental sample
MKSLFALSLFAGLSVAQNAAWAQCGGNGWTGSKTCVSGYKCTVVNEWYSQCIPGTAEEPTPTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 11 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaatactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 12 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLNTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 13 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtatgagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 14 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGMSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 15 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtcataatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 16 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGHNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 17 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtatgaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 18 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGMNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 19 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtgctgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 20 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGAEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 21 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcactttgacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 22 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITLTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 23 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcagagtctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 24 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSESDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 25 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaataactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 26 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWNNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 27 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctaccgctttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 28 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATAFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 29 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcatgagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 30 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFMSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 31 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaaggttaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcaaggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 32 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKVNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVKAATEWLRKNKKIGMIGELAGGPNDTCKTAVKNMLDYLKENSDVWKGVTWWAAGPWWADYMFSFEPPSGTGYQYYNSLLKTYI

SEQ ID NO: 33 - DNA, 1149 bp, Unknown
  Obtained from environmental sample
atgaagtccctcttcgctctcagcctcttcgccggcctttcggtcgctcaaaacgcagcatgggcccaatgcggtggaaacggctggactggctcaaagacctgcgtctctggctacaagtgcaccgtcgttaatgagtggtacagccagtgtatccccggtactgctgaggaacctactaccaccctcaagactactactggtggtggtagcacacctactggtacacctggaaatggaaagtttctctgggttggtactaatgaggctggtggtgagtttggtgagggcagtttgcctggaacttggggaaaacactttatcttccctgatcccgctgccgttgataccctcatctctcagggctacaacgccttccgcgttcaactccgcatggaacgcacaaaccctagctccatgaccggaccctttgacaccgcttacctcaagaacctcaccacaatcgtggaccacatcaccggcaagggcgccaacgtcattctcgaccctcacaactacggccgctactttgacaagatcatcacttcaacctctgacttccagacctggtggaagaactttgctacccagttcaagagcaacagcaaagtcatcttcgacactaacaatgagtacaacaccatggatcagactcttgttctgaacttgaaccaggctgctatcaacggtattcgtgctgctggcgctacccagactatttttgttgagggtaaccagtggtccggtgcttggtcttggcccgatgtcaacgacaacatgaaggctctcaccgaccctcttgacaagatcgtgtacgaaatgcaccagtacctcgactctgacagctccggtacttcacctaactgtgtctccaccaccattggagtcgagcgcgtcgcggctgctactgagtggttgaggaagaacaagaagattggcatgatcggtgaactcgccggcggtcctaacgacacctgcaagactgctgtcaagaatatgctggactatcttaaggagaactctgatgtttggaagggtgttacctggtgggctgctggtccttggtgggctgactacatgttcagctttgagcctcccagcggtactggttatcagtactacaactctcttctcaagacttatatctaa

SEQ ID NO: 34 - protein, 382 aa, Unknown
  Obtained from environmental sample
MKSLFALSLFAGLSVAQNAAWAQCGGNGWTGSKTCVSGYKCTVVNEWYSQCIPGTAEEPTTTLKTTTGGGSTPTGTPGNGKFLWVGTNEAGGEFGEGSLPGTWGKHFIFPDPAAVDTLISQGYNAFRVQLRMERTNPSSMTGPFDTAYLKNLTTIVDHITGKGANVILDPHNYGRYFDKIITSTSDFQTWWKNFATQFKSNSKVIFDTNNEYNTMDQTLVLNLNQAAINGIRAAGATQTIFVEGNQWSGAWSWPDVNDNMKALTDPLDKIVYEMHQYLDSDSSGTSPNCVSTTIGVERVAAATEWLRKNKKIGMIGELAGGPNDTCKTAVKNMLDYLKENSDVWKGVTWWAAGPWWADYMFSFEPPSGTGYQYYNSLLKTYI

(19) United States
(12) Patent Application Publication
(10) Pub. No.: US 2015/0010961 A1
(43) Pub. Date: Jan. 8, 2015

(54) POLYPEPTIDE HAVING ENDOGLUCANASE ACTIVITY

(71) Applicant: BP Corporation North America Inc., Houston, TX (US)

(72) Inventors: John Poland, San Diego, CA (US); Justin T. Stege, San Diego, CA (US)

(21) Appl. No.: 14/496,956

(22) Filed: Sep. 25, 2014

Related U.S. Application Data
(63) Continuation of application No. 13/840,091, filed on Mar. 15, 2013, now abandoned.
(60) Provisional application No. 61/611,765, filed on Mar. 16, 2012.

(51) Int. Cl. C12P 19/14; C12P 19/02; C12N 9/42

(52) U.S. Cl. CPC C12P 19/14; C12N 9/2437; C12P 19/02
USPC 435/99; 435/209; 435/320.1; 435/252.3; 435/348; 435/325; 435/254.11; 435/254.2; 435/419; 435/252.33; 435/254.21; 435/254.22; 435/254.3; 435/254.5; 435/254.9; 435/254.6; 435/254.7

(57) ABSTRACT

The present invention relates to a family 5 glycoside hydrolase variant having endoglucanase activity, polynucleotides encoding the family 5 glycoside hydrolase variant, vectors, host cells comprising the polynucleotides, and methods for using the family 5 glycoside hydrolase variant.

### CROSS-REFERENCE TO RELATED APPLICATIONS

This application is a continuation application of U.S. application Ser. No. 13/840,091 filed Mar. 15, 2013; which claims the benefit under 35 USC §119(e) to U.S. Application Ser. No. 61/611,765, filed Mar. 16, 2012, now expired The disclosure of each of the prior applications is considered part of and is incorporated by reference in the disclosure of this application.

### FIELD OF THE INVENTION

The present invention relates to a family 5 glycoside hydrolase variant having endoglucanase activity, polynucleotides encoding the family 5 glycoside hydrolase variant, vectors, host cells comprising the polynucleotides, and methods for using the family 5 glycoside hydrolase variant.

### BACKGROUND OF THE INVENTION

Cellulose is the structural component of the primary cell wall of plants, and may be one of the most abundant organic compounds found on earth. Cellulose is a polysaccharide polymer consisting of a linear chain of several β(1→4) linked glucose units. In order to access the individual glucose units the polysaccharide must be hydrolysed. This can be accomplished by the use of cellulase, a class of enzymes that catalyze the hydrolysis of cellulose.

Many microorganisms produce enzymes that hydrolyze β-linked glucans. These enzymes include endoglucanases, cellobiohydrolases, and β-glucosidases. Endoglucanases digest the cellulose polymer at random locations, opening it to attack by cellobiohydrolases.

Cellobiohydrolases sequentially release molecules of cellobiose from the ends of the cellulose polymer. Cellobiohydrolase I is a 1,4-D-glucan cellobiohydrolase activity which catalyzes the hydrolysis of 1,4-β-D-glucosidic linkages in cellulose, cellotetriose, or any beta-1,4-linked glucose containing polymer, releasing cellobiose from the reducing ends of the chain. Cellobiohydrolase II is a 1,4-D-glucan cellobiohydrolase activity which catalyzes the hydrolysis of 1,4-β-D-glucosidic linkages in cellulose, cellotetriose, or any β-1,4-linked glucose containing polymer, releasing cellobiose from the non-reducing ends of the chain. Cellobiose is a water-soluble β-1,4-linked dimer of glucose.

Beta-glucosidases hydrolyze cellobiose to glucose which can then be, among other things, fermented into ethanol.

It would be an advantage in the art to identify new endoglucanases having improved properties, such as improved hydrolysis rates, better thermal stability, reduced adsorption to lignin, and the ability to hydrolyze non-cellulosic components of biomass, such as hemicellulose, in addition to hydrolyzing cellulose. Endoglucanases with a broad range of side activities on hemicellulose can be especially beneficial for improving the overall hydrolysis yield of complex, hemicellulose-rich biomass substrates.

It is an object of the present invention to provide improved polypeptides having endoglucanase activity and polynucleotides encoding the polypeptides.

### SUMMARY OF THE INVENTION

The present invention provides polynucleotides and polypeptides encoded thereby which have been identified as endoglucanase enzymes having carboxymethyl cellulase activity (CMC).

In accordance with one aspect of the present invention, there is provided novel enzymes, as well as active analogs and fragments thereof.

In accordance with another aspect of the present invention, there are provided isolated polypeptides of the present invention as well as active fragments of such enzymes.

In accordance with yet a further aspect of the present invention, there is provided a process for producing such polypeptide by recombinant techniques comprising culturing recombinant prokaryotic and/or eukaryotic host cells, containing a nucleic acid sequence encoding an enzyme of the present invention, under conditions promoting expression of said enzyme and subsequent recovery of said enzyme.

In accordance with yet a further aspect of the present invention, there is provided a process for utilizing such enzymes, or polynucleotide encoding such enzymes for hydrolysis of cellulose. In accordance with yet a further aspect of the present invention, there is provided a process for utilizing such enzymes in the production of fuels.

These and other aspects of the present invention should be apparent to those skilled in the art from the teachings herein.

### BRIEF DESCRIPTION OF THE DRAWINGS

Further objects and features of the present invention will become more apparent from the following description of the invention, as shown in the accompanying drawings, in which:

FIG. 1 shows a schematic of how primary screens were run on the variants provided by this invention; and

FIG. 2 shows saccharification performance of the endoglucanase variants of the present invention.

### DETAILED DESCRIPTION OF THE INVENTION

In one embodiment, the present invention provides a family 5 glycoside hydrolase variant, the variant encoded by a mutated version of a wild-type parental polynucleotide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or complete sequence identity to a (cDNA) nucleotide sequence of SEQ ID NO: 1 and comprising at least one of the following nucleotide residue changes:

the nucleotides at positions 157 to 159 are TCT; (SEQ ID NO: 3)

the nucleotides at positions 175 to 177 are AAG; (SEQ ID NO: 5)

the nucleotides at positions 175 to 177 are CAT; (SEQ ID NO: 7)

the nucleotides at positions 181 to 183 are CCT; (SEQ ID NO: 9)

the nucleotides at positions 190 to 192 are AAT; (SEQ ID NO: 11)

the nucleotides at positions 208 to 210 are ATG; (SEQ ID NO: 13)

the nucleotides at positions 259 to 261 are CAT; (SEQ ID NO: 15)

the nucleotides at positions 259 to 261 are ATG; (SEQ ID NO: 17)

the nucleotides at positions 274 to 276 are GCT; (SEQ ID NO: 19)

the nucleotides at positions 547 to 549 are TTG; (SEQ ID NO: 21)

the nucleotides at positions 550 to 552 are GAG; (SEQ ID NO: 23)

the nucleotides at positions 574 to 576 are AAT; (SEQ ID NO: 25)

the nucleotides at positions 589 to 591 are GCT; (SEQ ID NO: 27)

the nucleotides at positions 595 to 597 are ATG; (SEQ ID NO: 29)

the nucleotides at positions 598 to 600 are GTT; (SEQ ID NO: 31) and

the nucleotides at positions 898 to 900 are GCG; (SEQ ID NO: 33)

wherein the variant has at least one of the following activities: endoglucanase activity, a beta-mannanase activity, an exo-1,3-glucanase activity, an endo-1,6-glucanase activity, a xylanase activity, and an endoglycoceramidase activity; and wherein the activity of the variant is greater than a polypeptide encoded by the wild-type parental polynucleotide having a nucleotide sequence of SEQ ID NO: 1.

In one embodiment, the present invention provides a family 5 glycoside hydrolase variant, wherein the family 5 glycoside hydrolase variant has endoglucanase activity.

In one embodiment, the present invention provides an expression cassette, a vector or a cloning vehicle comprising the mutated version of a wild-type parental polynucleotide sequence SEQ ID NO: 1 as set forth hereinabove, wherein optionally the cloning vehicle comprises a viral vector, a plasmid, a phage, a phagemid, a cosmid, a fosmid, a bacteriophage or an artificial chromosome, and optionally the viral vector comprises an adenovirus vector, a retroviral vector or an adeno-associated viral vector, and optionally the cloning vehicle comprises a bacterial artificial chromosome (BAC), a plasmid, a bacteriophage P1-derived vector (PAC), a yeast artificial chromosome (YAC), or a mammalian artificial chromosome (MAC).

In one embodiment, the present invention provides a transformed cell comprising a nucleic acid comprising the mutated version of a wild-type parental polynucleotide sequence SEQ ID NO: 1, or the expression cassette, the vector or the cloning vehicle as set forth hereinabove, wherein optionally the cell is a bacterial cell, a mammalian cell, a fungal cell, a yeast cell, an insect cell or a plant cell.

In one embodiment, the present invention provides a transformed cell comprising the expression vector as set forth hereinabove.

In one embodiment, the present invention provides a transformed cell, wherein the cell is a bacterial cell, a mammalian cell, a fungal cell, a yeast cell, an insect cell or a plant cell.

In one embodiment, the bacterial cell is selected from Zymomonas mobilis, Escherichia coli and Klebsiella oxytoca.

In one embodiment, the yeast cell is selected from Saccharomyces cerevisiae, Saccharomyces uvarum, Kluyveromyces fragilis, Kluyveromyces lactis, Candida pseudotropicalis, and Pachysolen tannophilus.

In one embodiment, the fungal cell is selected from the genus Aspergillus, Penicillium, Rhizopus, Chrysosporium, Myceliophthora, Trichoderma, Humicola, Acremonium or Fusarium

In one embodiment, the fungal cell is of the species Aspergillus niger, Aspergillus oryzae, Trichoderma reesei, Penicillium chrysogenum, Myceliophthora thermophila, or Rhizopus oryzae.

In one embodiment, the yeast cell is selected from the genus Saccharomyces, Kluyveromyces, Candida, Pichia, Schizosaccharomyces, Hansenula, Klockera, Schwanniomyces or Yarrowia.

In one embodiment, the yeast cell is of the species S. cerevisiae, S. bulderi, S. barnetti, S. exiguus, S. uvarum, S. diastaticus, K. lactis, K. marxianus or K. fragilis.

In one embodiment, the present invention provides a mature family 5 glycoside hydrolase variant, the variant having a mutated version of a wild-type parental polypeptide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or complete sequence identity to an amino acid sequence of SEQ ID NO: 2 and comprising at least one of the following amino acid residue changes:

proline is substituted with serine at position 53; (SEQ ID NO: 4)

proline is substituted with lysine at position 59; (SEQ ID NO: 6)

proline is substituted with histidine at position 59; (SEQ ID NO: 8)

threonine is substituted with proline at position 61; (SEQ ID NO: 10)

lysine is substituted with asparagine at position 64; (SEQ ID NO: 12)

glycine is substituted with methionine at position 70; (SEQ ID NO: 14)

threonine is substituted with histidine at position 87; (SEQ ID NO: 16)

threonine is substituted with methionine at position 87; (SEQ ID NO: 18)

glycine is substituted with alanine at position 92; (SEQ ID NO: 20)

serine is substituted with leucine at position 183; (SEQ ID NO: 22)

threonine is substituted with glutamic acid at position 184; (SEQ ID NO: 24)

lysine is substituted with asparagine at position 192; (SEQ ID NO: 26)

glutamine is substituted with alanine at position 197; (SEQ ID NO: 28)

lysine is substituted with methionine at position 199; (SEQ ID NO: 30)

serine is substituted with valine at position 200; (SEQ ID NO: 32) and

lysine is substituted with alanine at position 300; (SEQ ID NO: 34)

wherein the variant has at least one of the following activities: endoglucanase activity, a beta-mannanase activity, an exo-1,3-glucanase activity, an endo-1,6-glucanase activity, a xylanase activity, and an endoglycoceramidase activity; and wherein the activity of the variant is greater than the wild-type parental polypeptide having an amino acid sequence of SEQ ID NO: 2.

In one embodiment, the present invention provides a variant as set forth hereinabove, wherein the variant has endoglucanase activity.

In one embodiment, the present invention provides a method for making a fuel comprising a step of contacting a biomass with the polypeptide as set forth hereinabove.

In one embodiment, the biomass is at least one of the following: napier grass, energycane, sugarcane, sugarcane bagasse, sorghum, beets or sugarbeets, wheat, corn, soybeans, potato, rice or barley, switchgrass or Miscanthus, or any combination thereof.

In one embodiment, the fuel is at least one of the following: ethanol, methanol, propanol, butanol, or any combination thereof.

In one embodiment, the present invention provides a method for hydrolyzing cellulose comprising contacting biomass with a polypeptide as set forth hereinabove.

In one embodiment, the present invention provides a method wherein the biomass is subjected to a pretreatment process prior to being contacted with a polypeptide as set forth hereinabove.

In one embodiment, the present invention provides a method wherein the pretreatment process comprises the step of heating the biomass to at least 50° Celsius.

In one embodiment, the present invention provides a method wherein the pretreatment process comprises the step of contacting the biomass with an aqueous solution.

In one embodiment, the present invention provides a method wherein the aqueous solution has a pH of less than 7.

In one embodiment, the present invention provides a method wherein the aqueous solution has a pH of more than 7.

In one embodiment, the present invention provides an isolated mature family 5 glycoside hydrolase variant polypeptide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or complete sequence identity to of SEQ ID NO: 2 and comprising at least one of the following amino acid residue changes:

proline is substituted with serine at position 53; (SEQ ID NO: 4)

proline is substituted with lysine at position 59; (SEQ ID NO: 6)

proline is substituted with histidine at position 59; (SEQ ID NO: 8)

threonine is substituted with proline at position 61; (SEQ ID NO: 10)

lysine is substituted with asparagine at position 64; (SEQ ID NO: 12)

glycine is substituted with methionine at position 70; (SEQ ID NO: 14)

threonine is substituted with histidine at position 87; (SEQ ID NO: 16)

threonine is substituted with methionine at position 87; (SEQ ID NO: 18)

glycine is substituted with alanine at position 92; (SEQ ID NO: 20)

serine is substituted with leucine at position 183; (SEQ ID NO: 22)

threonine is substituted with glutamic acid at position 184; (SEQ ID NO: 24)

lysine is substituted with asparagine at position 192; (SEQ ID NO: 26)

glutamine is substituted with alanine at position 197; (SEQ ID NO: 28)

lysine is substituted with methionine at position 199; (SEQ ID NO: 30)

serine is substituted with valine at position 200; (SEQ ID NO: 32) and

lysine is substituted with alanine at position 300; (SEQ ID NO: 34)

wherein the isolated family 5 glycoside hydrolase variant has at least one of the following activities: endoglucanase activity, a beta-mannanase activity, an exo-1,3-glucanase activity, an endo-1,6-glucanase activity, a xylanase activity, and an endoglycoceramidase activity; and wherein the activity of the family 5 glycoside hydrolase variant is greater than its parent family 5 glycoside hydrolase encoded by the polynucleotide sequence of SEQ ID NO:1.

### DEFINITIONS

A DNA “coding sequence of” or a “nucleotide sequence encoding” a particular enzyme, is a DNA sequence which is transcribed and translated into an enzyme when placed under the control of appropriate regulatory sequences. The term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, or recombinant nucleotide sequence. The term “cDNA” is defined herein as a DNA molecule which can be prepared by reverse transcription from a mature, spliced, mRNA molecule obtained from a eukaryotic cell cDNA lacks intron sequences that are usually present in the corresponding genomic DNA. The initial, primary RNA transcript is a precursor to mRNA which is processed through a series of steps before appearing as mature spliced mRNA. These steps include the removal of intron sequences by a process called splicing. cDNA derived from mRNA lacks, therefore, any intron sequences.

The term “control sequences” is defined herein to include all components, which are necessary or advantageous for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “endoglucanase activity” is defined herein as an endo-1,4-beta-D-glucan 4-glucanohydrolase (E.C. No. 3.2.1.4) that catalyses the endohydrolysis of 1,4-beta-D-glycosidic linkages in cellulose, cellulose derivatives (such as carboxymethyl cellulose and hydroxyethyl cellulose), lignocellulose, lignocellulose derivatives, lichenin, beta-1,4 bonds in mixed beta-1,3 glucans such as cereal beta-D-glucans or xyloglucans, and other plant material containing cellulosic components. For purposes of the present invention, endoglucanase activity is determined using carboxymethyl cellulose (CMC) hydrolysis according to the procedure of Ghose, 1987, Pure and Appl. Chem. 59: 257-268.

The term “expression” includes any step involved in the production of the polypeptide including, but not limited to, transcription, post-transcriptional modification, translation, post-translational modification, and secretion.

The term “expression vector” is defined herein as a linear or circular DNA molecule that comprises a polynucleotide encoding a polypeptide of the present invention, and which is operably linked to additional nucleotides that provide for its expression.

The term “expression” refers to the process by which a polypeptide is produced based on the nucleic acid sequence of a gene. The process includes both transcription and translation.

“Family 5 glycoside hydrolase” or “Family GH5” comprise enzymes with several known activities; endoglucanase (EC:3.2.1.4); beta-mannanase (EC:3.2.1.78); exo-1,3-glucanase (EC:3.2.1.58); endo-1,6-glucanase (EC:3.2.1.75); xylanase (EC:3.2.1.8); endoglycoceramidase (EC:3.2.1.123).

The term “gene” means the segment of DNA involved in producing a polypeptide chain; it includes regions preceding and following the coding region (leader and trailer) as well as intervening sequences (introns) between individual coding segments (exons).

The term “host cell”, as used herein, includes any cell type which is susceptible to transformation, transfection, transduction, and the like with a nucleic acid construct or expression vector comprising a polynucleotide of the present invention.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

The term “isolated polypeptide” as used herein refers to a polypeptide which is free from other components from the organism from which it is derived. The term “isolated polypeptide” also covers polypeptides free from components from the native organism from which it is obtained. The polypeptide may be purified, with only minor amounts of other proteins being present. The term “purified” as used herein also refers to removal of other components, particularly other proteins and most particularly other enzymes present in the cell of origin of the enzyme of the invention. In one embodiment, the term “isolated polypeptide” refers to a polypeptide which is at least at least 75% (w/w), preferably at least 80%, more preferably at least 85%, more preferably at least 90%, more preferably at least 95%, more preferably at least 96%, more preferably at least 97%, even more preferably at least 98%, or most preferably at least 99% pure. In another preferred embodiment, the enzyme is 100% pure.

The term “mature polypeptide” is defined herein as a polypeptide that is in its final form following translation and any post-translational modifications, such as N-terminal processing, C-terminal truncation, glycosylation, no introns, and leader sequence cleaved, etc.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

The terms “transformed”, “stably transformed” or “transgenic” with reference to a cell means the cell has a non-native (heterologous) nucleic acid sequence integrated into its genome or as an episomal plasmid that is maintained through multiple generations.

In an effort to develop an effective minimum enzyme cocktail, a glycosyl hydrolase family 5 (GH5) fungal endo-1,4-β-D-glucanase was subjected to mutagenesis for improved activity and thermostability. A fungal GH5 endoglucanase was evolved using gene site specific mutagenesis (GSSM) technology for improved activity and thermotolerance on commercially relevant lignocellulosic substrates.

### Sources of Polypeptides Having Endoglucanase Activity

A polypeptide of the present invention may be obtained from microorganisms of any genus. For purposes of the present invention, the term “obtained from” as used herein in connection with a given source shall mean that the polypeptide encoded by a nucleotide sequence is produced by the source or by a strain in which the nucleotide sequence from the source has been inserted. In a preferred aspect, the polypeptide obtained from a given source is secreted extracellularly.

Furthermore, such polypeptides may be identified and obtained from other sources including microorganisms isolated from nature (e.g., soil, composts, water, etc.) using probes. Techniques for isolating microorganisms from natural habitats are well known in the art. The polynucleotide may then be obtained by similarly screening a genomic or cDNA library of such a microorganism. Once a polynucleotide sequence encoding a polypeptide has been detected with the probe(s), the polynucleotide can be isolated or cloned by utilizing techniques which are well known to those of ordinary skill in the art (see, e.g., Sambrook et al., 1989, supra).

### Polynucleotides

The present invention also relates to isolated polynucleotides comprising or consisting of a nucleotide sequence which encode a polypeptide of the present invention having endoglucanase activity.

### Cellulase Variants

The present invention provides new cellulase variants derived from a parental cellulase by substitution, insertion and/or deletion. A cellulase variant of this invention is a cellulase variant or mutated cellulase, having an amino acid sequence not found in nature. The cellulase variants of the invention show improved performance, in particular with respect to increased catalytic activity and/or altered thermostability.

Formally the cellulase variant or mutated cellulase of this invention may be regarded a functional derivative of a parental cellulase (i.e., the native or wild-type enzyme), and may be obtained by alteration of a DNA nucleotide sequence of the parental gene or its derivatives, encoding the parental enzyme. The cellulase variant or mutated cellulase may be expressed and produced when the DNA nucleotide sequence encoding the cellulase variant is inserted into a suitable vector in a suitable host organism. The host organism is not necessarily identical to the organism from which the parental gene originated.

### Modification of Nucleic Acids

The invention provides methods of generating variants of the nucleic acids of the invention, e.g., those encoding a glucanase (or cellulase), e.g., endoglucanase, mannanase, xylanase, amylase, xanthanase and/or glycosidase, e.g., cellobiohydrolase, mannanase and/or beta-glucosidase. These methods can be repeated or used in various combinations to generate glucanases, (or cellulases), e.g., endoglucanases, mannanases, xylanases, amylases, xanthanases and/or glycosidases, e.g., cellobiohydrolases, mannanases and/or beta-glucosidases having an altered or different activity or an altered or different stability from that of a glucanase (or cellulase), e.g., endoglucanase, mannanase, xylanase, amylase, xanthanase and/or glycosidase, e.g., cellobiohydrolase, mannanase and/or beta-glucosidase encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

In one aspect, the term “variant” refers to polynucleotides or polypeptides of the invention modified at one or more base pairs, codons, introns, exons, or amino acid residues (respectively) yet still retain the biological activity of a glucanase of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis (GSSM), synthetic ligation reassembly (SLR), and any combination thereof.

A nucleic acid of the invention can be altered by any means. For example, random or stochastic methods, or, non-stochastic, or “directed evolution,” methods, see, e.g., U.S. Pat. No. 6,361,974. Non-stochastic, or “directed evolution,” methods include, e.g., Gene Site Saturation Mutagenesis (GSSM), synthetic ligation reassembly (SLR), or a combination thereof are used to modify the nucleic acids of the invention to generate glucanases, (or cellulases), e.g., endoglucanases, mannanases, xylanases, amylases, xanthanases and/or glycosidases, e.g., cellobiohydrolases, mannanases and/or beta-glucosidases with new or altered properties (e.g., activity under highly acidic or alkaline conditions, high or low temperatures, and the like). Polypeptides encoded by the modified nucleic acids can be screened for an activity before testing for glucan or other polysaccharide hydrolysis or other activity. Any testing modality or protocol can be used, e.g., using a capillary array platform. See, e.g., U.S. Pat. Nos. 6,361,974; 6,280,926; 5,939,250.

### Saturation Mutagenesis, or, GSSM

The invention also provides methods for making new enzymes, or modifying sequences of the invention, using Gene Site Saturation mutagenesis, or, GSSM, as described herein, and also in U.S. Pat. Nos. 6,171,820, 6,765,835, 6,358,709, 6,562,594, 6,696,275, 6,764,835, 6,238,884, 6,773,900, 6,740,506 and 6,713,282.

In one aspect, codon primers containing a degenerate N,N,G/T sequence are used to introduce point mutations into a polynucleotide, e.g., a glucanase (or cellulase), e.g., endoglucanase, mannanase, xylanase, amylase, xanthanase and/or glycosidase, e.g., cellobiohydrolase, mannanase and/or beta-glucosidase or an antibody of the invention, so as to generate a set of progeny polypeptides in which a full range of single amino acid substitutions is represented at each amino acid position, e.g., an amino acid residue in an enzyme active site (catalytic domains (CDs)) or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position×100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide (e.g., glucanases, (or cellulases), e.g., endoglucanases, mannanases, xylanases, amylases, xanthanases and/or glycosidases, e.g., cellobiohydrolases, mannanases and/or beta-glucosidases) molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased glucan hydrolysis activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In yet another aspect, site-saturation mutagenesis can be used together with shuffling, chimerization, recombination and other mutagenizing processes, along with screening. This invention provides for the use of any mutagenizing process(es), including saturation mutagenesis, in an iterative manner. In one exemplification, the iterative use of any mutagenizing process(es) is used in combination with screening.

The invention also provides for the use of proprietary codon primers (containing a degenerate N,N,N sequence) to introduce point mutations into a polynucleotide, so as to generate a set of progeny polypeptides in which a full range of single amino acid substitutions is represented at each amino acid position (Gene Site Saturation Mutagenesis (GSSM)). The oligos used are comprised contiguously of a first homologous sequence, a degenerate N,N,N sequence and in one aspect but not necessarily a second homologous sequence. The downstream progeny translational products from the use of such oligos include all possible amino acid changes at each amino acid site along the polypeptide, because the degeneracy of the N,N,N sequence includes codons for all 20 amino acids.

In one aspect, one such degenerate oligo (comprised of one degenerate N,N,N cassette) is used for subjecting each original codon in a parental polynucleotide template to a full range of codon substitutions. In another aspect, at least two degenerate N,N,N cassettes are used—either in the same oligo or not, for subjecting at least two original codons in a parental polynucleotide template to a full range of codon substitutions. Thus, more than one N,N,N sequence can be contained in one oligo to introduce amino acid mutations at more than one site. This plurality of N,N,N sequences can be directly contiguous, or separated by one or more additional nucleotide sequence(s). In another aspect, oligos serviceable for introducing additions and deletions can be used either alone or in combination with the codons containing an N,N,N sequence, to introduce any combination or permutation of amino acid additions, deletions and/or substitutions.

In a particular exemplification, it is possible to simultaneously mutagenize two or more contiguous amino acid positions using an oligo that contains contiguous N,N,N triplets, i.e. a degenerate (N,N,N)ₙ sequence.

In another aspect, the present invention provides for the use of degenerate cassettes having less degeneracy than the N,N,N sequence. For example, it may be desirable in some instances to use (e.g. in an oligo) a degenerate triplet sequence comprised of only one N, where the N can be in the first second or third position of the triplet. Any other bases including any combinations and permutations thereof can be used in the remaining two positions of the triplet. Alternatively, it may be desirable in some instances to use (e.g., in an oligo) a degenerate N,N,N triplet sequence, N,N,G/T, or an N,N, G/C triplet sequence.

It is appreciated, however, that the use of a degenerate triplet (such as N,N,G/T or an N,N, G/C triplet sequence) as disclosed in the instant invention is advantageous for several reasons. In one aspect, this invention provides a means to systematically and fairly easily generate the substitution of the full range of possible amino acids (for a total of 20 amino acids) into each and every amino acid position in a polypeptide. Thus, for a 100 amino acid polypeptide, the invention provides a way to systematically and fairly easily generate 2000 distinct species (i.e., 20 possible amino acids per position times 100 amino acid positions). It is appreciated that there is provided, through the use of an oligo containing a degenerate N,N,G/T or an N,N, G/C triplet sequence, 32 individual sequences that code for 20 possible amino acids. Thus, in a reaction vessel in which a parental polynucleotide sequence is subjected to saturation mutagenesis using one such oligo, there are generated 32 distinct progeny polynucleotides encoding 20 distinct polypeptides. In contrast, the use of a non-degenerate oligo in site-directed mutagenesis leads to only one progeny polypeptide product per reaction vessel.

This invention also provides for the use of nondegenerate oligos, which can optionally be used in combination with degenerate primers disclosed. It is appreciated that in some situations, it is advantageous to use nondegenerate oligos to generate specific point mutations in a working polynucleotide. This provides a means to generate specific silent point mutations, point mutations leading to corresponding amino acid changes and point mutations that cause the generation of stop codons and the corresponding expression of polypeptide fragments.

Thus, in one aspect of this invention, each saturation mutagenesis reaction vessel contains polynucleotides encoding at least 20 progeny polypeptide molecules such that all 20 amino acids are represented at the one specific amino acid position corresponding to the codon position mutagenized in the parental polynucleotide. The 32-fold degenerate progeny polypeptides generated from each saturation mutagenesis reaction vessel can be subjected to clonal amplification (e.g., cloned into a suitable E. coli host using an expression vector) and subjected to expression screening. When an individual progeny polypeptide is identified by screening to display a favorable change in property (when compared to the parental polypeptide), it can be sequenced to identify the correspondingly favorable amino acid substitution contained therein.

It is appreciated that upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid and each of two favorable changes) and 3 positions. Thus, there are 3×3×3 or 27 total possibilities, including 7 that were previously examined—6 single point mutations (i.e., 2 at each of three positions) and no change at any position.

Thus, in a non-limiting exemplification, this invention provides for the use of saturation mutagenesis in combination with additional mutagenization processes, such as process where two or more related polynucleotides are introduced into a suitable host cell such that a hybrid polynucleotide is generated by recombination and reductive reassortment.

In addition to performing mutagenesis along the entire sequence of a gene, the instant invention provides that mutagenesis can be use to replace each of any number of bases in a polynucleotide sequence, wherein the number of bases to be mutagenized is in one aspect every integer from 15 to 100,000. Thus, instead of mutagenizing every position along a molecule, one can subject every or a discrete number of bases (in one aspect a subset totaling from 15 to 100,000) to mutagenesis. In one aspect, a separate nucleotide is used for mutagenizing each position or group of positions along a polynucleotide sequence. A group of 3 positions to be mutagenized may be a codon. The mutations can be introduced using a mutagenic primer, containing a heterologous cassette, also referred to as a mutagenic cassette. Exemplary cassettes can have from 1 to 500 bases. Each nucleotide position in such heterologous cassettes be N, A, C, G, T, A/C, A/G, A/T, C/G, C/T, G/T, C/G/T, A/G/T, A/C/T, A/C/G, or E, where E is any base that is not A, C, G, or T (E can be referred to as a designer oligo).

In one aspect, saturation mutagenesis comprises mutagenizing a complete set of mutagenic cassettes (wherein each cassette is in one aspect about 1-500 bases in length) in defined polynucleotide sequence to be mutagenized (wherein the sequence to be mutagenized is in one aspect from about 15 to 100,000 bases in length). Thus, a group of mutations (ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons and groupings of particular nucleotide cassettes.

Defined sequences to be mutagenized include a whole gene, pathway, cDNA, an entire open reading frame (ORF) and entire promoter, enhancer, repressor/transactivator, origin of replication, intron, operator, or any polynucleotide functional group. Generally, a “defined sequences” for this purpose may be any polynucleotide that a 15 base-polynucleotide sequence and polynucleotide sequences of lengths between 15 bases and 15,000 bases (this invention specifically names every integer in between). Considerations in choosing groupings of codons include types of amino acids encoded by a degenerate mutagenic cassette.

In one exemplification a grouping of mutations that can be introduced into a mutagenic cassette, this invention specifically provides for degenerate codon substitutions (using degenerate oligos) that code for 2, 3, 4, 5, 6, 7, 8, 9, 10, 11, 12, 13, 14, 15, 16, 17, 18, 19 and 20 amino acids at each position and a library of polypeptides encoded thereby.

### Optimized Directed Evolution System

The invention provides a non-stochastic gene modification system termed “optimized directed evolution system” to generate polypeptides, e.g., glucanases, (or cellulases), e.g., endoglucanases, mannanases, xylanases, amylases, xanthanases and/or glycosidases, e.g., cellobiohydrolases, mannanases and/or beta-glucosidases or antibodies of the invention, with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide in one aspect includes a unique region of overlap so that mixing the oligonucleotides together results in a new variant that has each oligonucleotide fragment assembled in the correct order. Alternatively protocols for practicing these methods of the invention can be found in U.S. Pat. Nos. 6,773,900; 6,740,506; 6,713,282; 6,635,449; 6,605,449; 6,537,776; 6,361,974.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a 1/3 chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide in one aspect includes a unique region of overlap so that mixing the oligonucleotides together results in a new variant that has each oligonucleotide fragment assembled in the correct order. See also U.S. Pat. Nos. 6,537,776; 6,605,449.

The evolved endoglucanase could be used as part of a novel minimum cellulase cocktail or could supplement or replace the endoglucanase component of a full fungal cellulase mixture in saccharification or simultaneous saccharification and fermentation (SSF) reactions for ethanol production from biomass.

Individual point mutations were introduced into the GH5 Endoglucanase, BD25243, by GSSM and expressed in A. niger. Expressed enzyme variants were screened on ground, steam exploded bagasse under process relevant conditions for improved specific activity. The top mutations identified are listed below in Table 1.

TABLE 1

| | Percent (%) | Percent (%) | | |
|---|---|---|---|---|
| | Improvement | Improvement | Percent (%) | |
| | over | over | Improvement | |
| SEQ | Wild-Type* | Wild-Type* | over Wild-Type* | Dose |
| ID NOS: | at 24 Hours | at 48 Hours | at 72 Hours | Reduction |
| 3, 4 | 15.3 | 24.2 | 10.6 | 0.75 |
| 5, 6 | 16.2 | 13.0 | — | 0.60 |
| 7, 8 | 25.1 | 16.9 | — | 0.65 |
| 9, 10 | 11.0 | 11.3 | 12.3 | 0.50 |
| 11, 12 | 8.8 | 7.8 | 11.6 | 0.50 |
| 13, 14 | 9.9 | 10.9 | 3.7 | 0.75 |
| 15, 16 | 2.5 | 9.6 | 2.7 | 0.70 |
| 17, 18 | 6.6 | 5.9 | 3.2 | 0.75 |
| 19, 20 | 9.2 | 7.5 | 10.2 | 0.50 |
| 21, 22 | 10.5 | 9.5 | 13.2 | 0.75 |
| 23, 24 | 14.8 | 13.5 | 12.9 | 0.60 |
| 25, 26 | 17.5 | 27.2 | 12.8 | 0.50 |
| 27, 28 | 8.7 | 10.1 | 9.9 | 0.60 |
| 29, 30 | 14.7 | 25.1 | 13.3 | 0.50 |
| 31, 32 | 8.9 | 8.6 | 12.6 | 0.70 |
| 33, 34 | 10.5 | 13.1 | 13.1 | 0.80 |

*SEQ ID NOS: 1, 2

### EXAMPLES

### Example 1

### GSSM (Gene Site-Saturated Mutagenesis) of the Wild-Type Endoglucanase

Overlapping DNA primers containing NNK degeneracy, where N represents any nucleotide (A, C, G, or T) and where K represents the keto group containing nucleotides (G or T), were used to create a library of variants for every amino acid position in the gDNA sequence of the endoglucanase (EG) between the end of the signal peptide and the stop codon (360 residues). The mutated residues included the complete N-terminal CBM domain, the linker region, and the catalytic domain. The NNK degeneracy of the mutagenesis primers can potentially generate 32 different codons covering all 20 possible amino acids at each residue.

GSSM reactions were run in 96-well plates using methylated template DNA of the wild-type EG prepared from a standard laboratory dam+E. coli host strain. Paired forward and reverse NNK degenerate primers for each amino acid position were combined with the template DNA along with dNTPs, reaction buffer and high fidelity DNA polymerase. GSSM reactions were run under standard PCR conditions, with elongation times appropriate for amplification of the protein of interest and the replicating plasmid on which it was contained. Each GSSM reaction produced products consisting of a library of variants, potentially containing up to all 20 possible amino acids, for a single residue. The reaction products were treated with DpnI restriction enzyme to digest the methylated wild-type template DNA and leave the non-methylated variant DNA intact. After DpnI treatment the PCR products were run on a 1% agarose gel and stained with ethidium bromide to confirm amplification of the plasmid.

In this instance, a “Sequence First” approach was used, whereby successfully amplified plasmid libraries were transformed into a standard laboratory E. coli strain, and the resulting isolated colonies, each containing a single library variant, were used to inoculate cultures in 384-well plates. After overnight growth, a duplicate plate was generated for archiving and DNA from the cultures was used for sequencing to determine the nucleotide mutations, and thereby the resulting amino acid mutation, introduced by the degenerate primers. This sequencing step also provided a QC step to eliminate colonies containing wild-type sequences or unwanted mutations (undirected point mutations, additions, or deletions). If fewer than sixteen amino acids variants (not including the wild-type) were obtained from a GSSM reaction, a second 384-well plate of isolated colonies could be picked for additional sequencing. In rare instances with low amino acid coverage the GSSM reaction was repeated with the original NNK primers, or occasionally with primers containing the minimal degeneracy needed to obtain the amino acids not found in sequencing of the original GSSM reaction.

After sequencing, mutations for every amino acid obtained by the GSSM reaction were cherry picked in duplicate from the archive plate into a 96-well culture plate. Where possible the duplicate amino acid variants for a single residue consisted of two different nucleotide variants encoding the same amino acid. Typically amino acid variants for two residues were cherry picked into each half of a plate. These cultures were grown overnight and archived for use in downstream processing. In this case the genes of known sequence would be transformed into a fungal screening host.

### Example 2

The parent EG gene was inserted into the pDC-A2 vector and the variants were made using GSSM technology. The library was transformed into E. coli Stbl2, sequenced, and then passed on for fungal transformations into Aspergillus niger.

The pDC-A2 vector used in making the EG variants of the invention was a reconstruction of the vector pGBFin-5 (described, e.g., in U.S. Pat. No. 7,220,542), which was remade to reduce the total size of the vector. The 2.1 kb 3′ Gla region of pGBFin-5 was reduced to 0.54 kb, the gpd promoter remained the same, but the 2.24 kb amdS sequence was replaced by the 1.02 kb hygB gene encoding hygromycin phosphotransferase. The 2.3 kb 3′ Gla region of pGBFin-5 was reduced to a 1.1 kb fragment representing the 5′ end of the original sequence. The E. coli replicon for pDC-A2 was taken from pUC18.

After transformation of the vector into E. coli Stbl2, individual E. coli transformants were picked into 96-well plates and grown in liquid culture in 200 μl LB plus ampicillin (100 μg/ml) per well overnight at 30° C. The cells were then used to generate template for sequencing reactions by colony PCR. The sequence data from the library of clones was analyzed to identify unique variants of EG. The E. coli transformants containing the selected variants were then rearrayed in 96-well format and used to prepare linear DNA of the entire expression cassette (the contents of pDC-A2 with the exception of the E. coli replicon) by PCR, using primers hybridizing to the ends of the 3′ and 3″ Gla regions. Approximately 1 μg of PCR product from each clone was then used to transform A. niger protoplasts in a PEG-mediated transformation in one well of a 96-well plate (i.e. one clone per well). Transformants were selected on regeneration agar (200 μl per well of PDA plus sucrose at 340 g/l and hygromycin at 200 μg/ml) in the same 96-well format. After 7 days incubation at 30° C., transformants were replicated to 96-well plates containing PDA plus hygromycin (200 μg/ml) using a pintool. Following incubation at 30° C. for a further 7 days, spores from each well were used to inoculate 200 μl liquid media per well of a 96-well plate. The plates were incubated at 30° C. for 7 days, and the supernatant from each well, containing the secreted EG variant, was recovered.

The media used to grow the Aspergillus transformed with expression constructs containing the variants had the following composition: NaNO₃, 3.0 g/l; KCl, 0.26 g/l; KH₂PO₄, 0.76 g/1; 4M KOH, 0.56 ml/l; D-Glucose, 5.0 g/l; Casamino Acids, 0.5 g/l; Trace Element Solution 0.5 m/l; Vitamin Solution 5 m/l; Penicillin-Streptomycin Solution (10,000 U/ml and 10,000 μg/ml respectively) 5.0 m/l; Maltose, 66.0 g/l; Soytone, 26.4 g/l; (NH₄)₂SO₄, 6.6 g/l; NaH₂PO₄.H₂O, 0.44 g/l; MgSO₄.7H₂O, 0.44 g/l; Arginine, 0.44 g/l; Tween-80, 0.035 m/l; Pleuronic Acid Antifoam, 0.0088 m/l; MES, 18.0 g/l. The Trace Element Solution had the following composition in 100 ml: ZnSO₄.7H₂O, 2.2 g; H₃BO₃, 1.1 g; FeSO₄.7H₂O, 0.5 g; CoCl₂.6H₂O, 0.17 g; CuSO₄.5H₂O, 0.16; MnCl₂.4H₂O, 0.5 g/l; NaMoO₄.2H₂O, 0.15 g/l; EDTA, 5 g/l. The Vitamin Solution had the following composition in 500 ml: Riboflavin, 100 mg; Thiamine.HCl, 100 mg; Nicotinamide, 100 mg; Pyridoxine.HCl, 50 mg; Panthotenic Acid, 10 mg; Biotin 0.2 mg.

### Example 3

### Assay Conditions for EG GSSM Screening

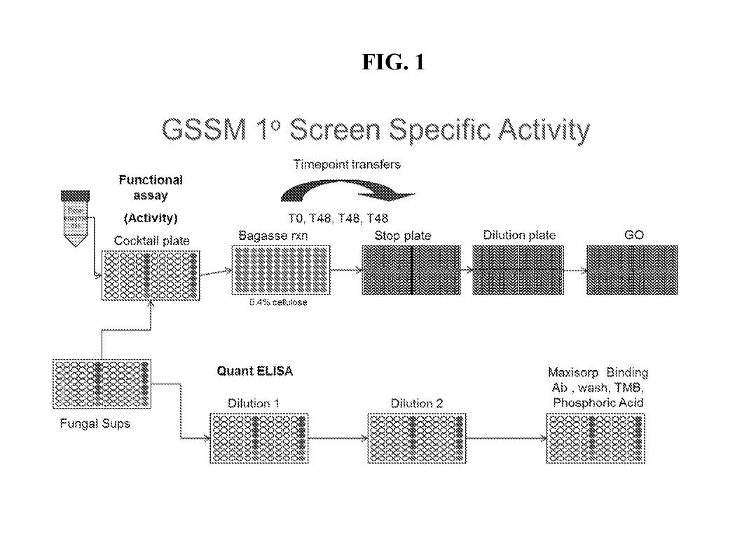

Endoglucanase GSSM mutants were grown in liquid cultures by transferring fungal transformation spores from agar plates into liquid media in 96-well Pall filter plates. After 5-7 days of growth the cultures were harvested by centrifugation into a new 96-well plate in order to filter the supernatants away from the fungal biomass prior to screening. Each supernatant plate typically contained the unique amino acid variants for two GSSM residues along with two columns of wild-type and vector only controls. Wild-type controls would include both supernatants from transformants grown on the plate and added into the plate after harvest from independently grown cultures. This independent culture would be pre-quantified by gel densitometry and the “spiked-in” controls added at three known concentrations to create a dose response curve. Supernatants were then split into two streams for two separate high throughput screens, shown in FIG. 1. One screen detected activity through enzymatic digestion of pretreated biomass, measured by glucose release, and the other was an enzyme quantitation ELISA done with a protein specific Ab.

The activity assay measured the glucose released from digestion of acid pre-treated, steam exploded bagasse. The pretreated substrate was washed, dried and milled to 40-mesh, followed by compositional analysis using standard methods. This substrate was used to prepare a 0.4% cellulose slurry in 50 mM NaOAC pH 5.0 buffer and dispensed into 96-well plates. Enzyme cocktail plates were created using wild-type CBHI and CBHII, and the EG mutant supernatants. The inclusion of these enzymes is important in that all three act synergistically to digest bagasse. This cocktail was added to the substrate plates to initiate the reaction. Samples were mixed thoroughly and then centrifuged before transferring an initial time point into a 384-well stop plate containing 400 mM NaCarbonate pH10 buffer. The reaction plates were sealed and placed in a shaking incubator at 37° C. for 48 hours, after which they were centrifuged again and three replicate 48 hr time points were transferred into the stop plate. A glucose oxidase assay, which measures the signal of monomeric glucose, was performed on the timepoints to measure the enzymatic release of glucose from the substrate. Samples from the stop plate were added to a mix containing Sodium Phosphate pH7.4 buffer, Glucose Oxidase (Sigma #G7141-50KU), Horseradish Peroxidase (Sigma #P2088-5KU), and Amplex red (Invitrogen No. 22177). This mixture was incubated at room temp for 30 minutes and the fluorescence measured at excitation/emission 560/610 nm.

The ELISA quant assay measured the concentration of expressed protein with enzyme specific polyclonal antibodies produced in rabbits inoculated with purified wild-type EG. Fungal expressed proteins were diluted in PBS and transferred to NUNC Immuno Maxisorp plates for overnight binding. The next day blocking reagent was added to the samples followed by subsequent incubations with the optimized dilutions of 1° antibody and 2° antibody (Sigma anti-rabbit whole molecule grown in goat with Peroxidase). A SureBlue TMB detection reagent was added, followed by a stop reagent (1M phosphoric acid) and the absorbance was read at 450 nm.

Bagasse activity and ELISA data were analyzed to calculate the specific activity of each variant compared to the wild-type controls. Samples that showed higher specific activity than the controls were selected for secondary screening.

For the 2° screen primary hits were cherry picked in quadruplicate from frozen archived fungal spore plates and regrown in liquid culture in 96-well PALL plates for supernatant harvest. The activity and ELISA assays described above were performed on these supernatants. The data were analyzed to select hits for tertiary screening.

### Example 4

### Tertiary and Dose Response Screening

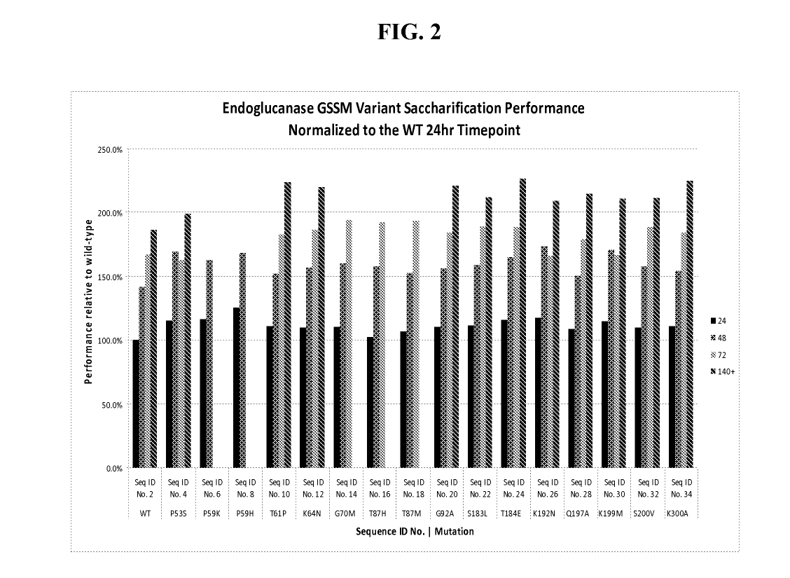

Secondary hits were grown up in 50 mL shake flask A. niger cultures. After harvesting and recovery, protein concentrations for hits were determined using gel densitometry versus a purified wild-type EG standard to determine precise concentrations. Tertiary screen reactions were performed in 10 ml vials in duplicate at 35° C. The reaction volume was 5 ml, with steam-exploded bagasse loaded at 5% solids in 50 mM sodium acetate pH5.2 and 1 mM sodium azide to prevent contamination. Two stainless steel BBs were added to each vial, which were then capped and sealed, and placed in a shaking incubator at 300 rpm. EG variants were compared to wild type EG in the presence of wild-type CBHI and CBHII. The total enzyme dose was 10 mg enzyme/g cellulose with a 2:2:1 ratio (EG:CBHI:CBHII). Timepoints were taken at TO, 24, 48, and 72 hours and analyzed by HPLC using refractive index detection (RID) to measure sugar products (glucose, cellobiose etc). The results are shown in FIG. 2 and Table 2.

Top performing hits from the tertiary screen were tested in a dose response assay following a very similar format, except that the EG variants were loaded at 1×, 0.75×, and 0.5× concentration, while holding the CBHI and CBHII dose constant. The purpose of the dose response assay was to determine the dose reduction allowed by the EG variants that yielded the same performance as the 1× wild-type EG dose.

TABLE 2
Normalized to WT 24 hour time point

| Mutation | SEQ ID NO | 24 hr | 48 hr | 72 hr | 140+ hr |
|---|---|---|---|---|---|
| WT | 2 | 100.0% | 141.4% | 167.1% | 186.2% |
| P53S | 4 | 115.3% | 169.5% | 163.0% | 198.9% |
| P59K | 6 | 116.2% | 162.9% | | |
| P59H | 8 | 125.1% | 168.5% | | |
| T61P | 10 | 111.0% | 152.2% | 182.7% | 223.8% |
| K64N | 12 | 109.6% | 156.4% | 186.5% | 220.0% |
| G70M | 14 | 109.9% | 159.9% | 194.2% | |
| T87H | 16 | 102.5% | 158.0% | 192.5% | |
| T87M | 18 | 106.6% | 152.7% | 193.4% | |
| G92A | 20 | 109.9% | 156.1% | 184.2% | 221.1% |
| S183L | 22 | 111.2% | 159.0% | 189.2% | 212.1% |
| T184E | 24 | 115.6% | 164.8% | 188.7% | 226.3% |
| K192N | 26 | 117.5% | 173.6% | 166.2% | 209.1% |
| Q197A | 28 | 108.7% | 150.6% | 178.7% | 214.5% |
| K199M | 30 | 114.7% | 170.7% | 166.9% | 211.0% |
| S200V | 32 | 109.6% | 157.7% | 188.3% | 211.4% |
| K300A | 34 | 110.5% | 154.6% | 184.0% | 224.8% |

Sequence Listing
SEQ. ID. NO. 1
(Wild Type)
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 2
(Wild Type)
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 3
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCTCTGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 4
QNAAWAQCGGNGWTGSKTCVSGYKCTVVNEWYSQCISGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 5
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAAAAGACTACCACCCTCAAGACTACTACTGGTGGTGGTAGCACACCTACTGGTA
CACCTGGAAATGGAAAGTTTCTCTGGGTTGGTACTAATGAGGCTGGTGGTGAGTTTG
GTGAGGGCAGTTTGCCTGGAACTTGGGGAAAACACTTTATCTTCCCTGATCCCGCTG
CCGTTGATACCCTCATCTCTCAGGGCTACAACGCCTTCCGCGTTCAACTCCGCATGG
AACGCACAAACCCTAGCTCCATGACCGGACCCTTTGACACCGCTTACCTCAAGAACC
TCACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 6
QNAAWAQCGGNGWTGSKTCVSGYKCTVVNEWYSQCIPGTAEEKTTTLKTTTGGGSTP
TGTPGNGKFLWVGTNEAGGEFGEGSLPGTWGKEFIFPDPAAVDTLISQGYNAFRVQLR
MERTNPSSMTGPFDTAYLKNLTTIVDHITGKGANVILDPHNYGRYFDKIITSTSDFQTWW
KNFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 7
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACATACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 8
QNAAWAQCGGNGWTGSKTCVSGYKCTVVNEWYSQCIPGTAEEHTTTLKTTTGGGSTP
TGTPGNGKFLWVGTNEAGGEFGEGSLPGTWGKHFIFPDPAAVDTLISQGYNAFRVQLR
MERTNPSSMTGPFDTAYLKNLTTIVDHITGKGANVILDPHNYGRYFDKIITSTSDFQTWW
KNFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 9
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTCCTACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 10
QNAAWAQCGGNGWTGSKTCVSGYKCTVVNEWYSQCIPGTAEEPTPTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 11
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAATACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 12
QNAAWAQCGGNGWTGSKTCVSGYKCTVVNEWYSQCIPGTAEEPTTTLNTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 13
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTATGAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 14
QNAAWAQCGGNGWTGSKTCVSGYKCTVVNEWYSQCIPGTAEEPTTTLKTTTGGMSTP
TGTPGNGKFLWYGTNEAGGEFGEGSLPGTWGKHFIFPDPAAVDTLISQGYNAFRVQLR
MERTNPSSMTGPFDTAYLKNLTTIVDHITGKGANVILDPHNYGRYFDKIITSTSDFQTWW
KNFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 15
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTCATAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 16
QNAAWAQCGGNGWTGSKTCVSGYKCTVVNEWYSQCIPGTAEEPTTTLKTTTGGGSTPT
GTPGNGKFLWVGHNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 17
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTATGAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 18
QNAAWAQCGGNGWTGSKTCVSGYKCTVVNEWYSQCIPGTAEEPTTTLKTTTGGGSTPT
GTPGNGKFLWVGMNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 19
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGCTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 20
QNAAWAQCGGNGWTGSKTCVSGYKCTVVNEWYSQCIPGTAEEPTTTLKTTTGGGSTPT
GTPGNGKFLWVGTNEAGAEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 21
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTTGACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 22
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITLTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 23
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAGAGTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCAAGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 24
QNAAWAQCGGNGWTGSKTCVSGYKCTVVNEWYSQCIPGTAEEPTTTLKTTTGGGSTPT
GTPGNGKFLWVGTNEAGGEFGEGSLPGTWOKHFIFPDPAAVDTLISQGYIAFRVQLRM
ERTNPSSMTGPFDTAYLKNLTTIVDHITGKGANVILDPHNYGRYFDKIITSESDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 25
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAATAACTTTGCTACCCAGTTCAAGAGCAACAGCAAAGTCATCTTCGACACTA
ACAATGAGTACAACACCATGGATCAGACTCTTGTTCTGAACTTGAACCAGGCTGCTA
TCAACGGTATTCGTGCTGCTGGCGCTACCCAGACTATTTTTGTTGAGGGTAACCAGT
GGTCCGGTGCTTGGTCTTGGCCCGATGTCAACGACAACATGAAGGCTCTCACCGACC
CTCTTGACAAGATCGTGTACGAAATGCACCAGTACCTCGACTCTGACAGCTCCGGTA
CTTCACCTAACTGTGTCTCCACCACCATTGGAGTCGAGCGCGTCAAGGCTGCTACTG
AGTGGTTGAGGAAGAACAAGAAGATTGGCATGATCGGTGAACTCGCCGGCGGTCCT
AACGACACCTGCAAGACTGCTGTCAAGAATATGCTGGACTATCTTAAGGAGAACTCT
GATGTTTGGAAGGGTGTTACCTGGTGGGCTGCTGGTCCTTGGTGGGCTGACTACATG
TTCAGCTTTGAGCCTCCCAGCGGTACTGGTTATCAGTACTACAACTCTCTTCTCAAGA
CTTATATCTAA
SEQ. ID. NO. 26
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWN
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 27
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCGCTTTCAAGAGCAACAGCAAAGTCATCTTCGACACTA
ACAATGAGTACAACACCATGGATCAGACTCTTGTTCTGAACTTGAACCAGGCTGCTA
TCAACGGTATTCGTGCTGCTGGCGCTACCCAGACTATTTTTGTTGAGGGTAACCAGT
GGTCCGGTGCTTGGTCTTGGCCCGATGTCAACGACAACATGAAGGCTCTCACCGACC
CTCTTGACAAGATCGTGTACGAAATGCACCAGTACCTCGACTCTGACAGCTCCGGTA
CTTCACCTAACTGTGTCTCCACCACCATTGGAGTCGAGCGCGTCAAGGCTGCTACTG
AGTGGTTGAGGAAGAACAAGAAGATTGGCATGATCGGTGAACTCGCCGGCGGTCCT
AACGACACCTGCAAGACTGCTGTCAAGAATATGCTGGACTATCTTAAGGAGAACTCT
GATGTTTGGAAGGGTGTTACCTGGTGGGCTGCTGGTCCTTGGTGGGCTGACTACATG
TTCAGCTTTGAGCCTCCCAGCGGTACTGGTTATCAGTACTACAACTCTCTTCTCAAGA
CTTATATCTAA
SEQ. ID. NO. 28
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATAFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 29
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCATGAGCAACAGCAAAGTCATCTTCGACACTA
ACAATGAGTACAACACCATGGATCAGACTCTTGTTCTGAACTTGAACCAGGCTGCTA
TCAACGGTATTCGTGCTGCTGGCGCTACCCAGACTATTTTTGTTGAGGGTAACCAGT
GGTCCGGTGCTTGGTCTTGGCCCGATGTCAACGACAACATGAAGGCTCTCACCGACC
CTCTTGACAAGATCGTGTACGAAATGCACCAGTACCTCGACTCTGACAGCTCCGGTA
CTTCACCTAACTGTGTCTCCACCACCATTGGAGTCGAGCGCGTCAAGGCTGCTACTG
AGTGGTTGAGGAAGAACAAGAAGATTGGCATGATCGGTGAACTCGCCGGCGGTCCT
AACGACACCTGCAAGACTGCTGTCAAGAATATGCTGGACTATCTTAAGGAGAACTCT
GATGTTTGGAAGGGTGTTACCTGGTGGGCTGCTGGTCCTTGGTGGGCTGACTACATG
TTCAGCTTTGAGCCTCCCAGCGGTACTGGTTATCAGTACTACAACTCTCTTCTCAAGA
CTTATATCTAA
SEQ. ID. NO. 30
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFMSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 31
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGGTTAACAGCAAAGTCATCTTCGACACTA
ACAATGAGTACAACACCATGGATCAGACTCTTGTTCTGAACTTGAACCAGGCTGCTA
TCAACGGTATTCGTGCTGCTGGCGCTACCCAGACTATTTTTGTTGAGGGTAACCAGT
GGTCCGGTGCTTGGTCTTGGCCCGATGTCAACGACAACATGAAGGCTCTCACCGACC
CTCTTGACAAGATCGTGTACGAAATGCACCAGTACCTCGACTCTGACAGCTCCGGTA
CTTCACCTAACTGTGTCTCCACCACCATTGGAGTCGAGCGCGTCAAGGCTGCTACTG
AGTGGTTGAGGAAGAACAAGAAGATTGGCATGATCGGTGAACTCGCCGGCGGTCCT
AACGACACCTGCAAGACTGCTGTCAAGAATATGCTGGACTATCTTAAGGAGAACTCT
GATGTTTGGAAGGGTGTTACCTGGTGGGCTGCTGGTCCTTGGTGGGCTGACTACATG
TTCAGCTTTGAGCCTCCCAGCGGTACTGGTTATCAGTACTACAACTCTCTTCTCAAGA
CTTATATCTAA
SEQ. ID. NO. 32
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKHTSTSDFQTWWK
NFATQFKVNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVKAATEWLR
KNKKIGMIGELAGGPNDTCKTAVKNMLDYLKENSDVWKGVTWWAAGPWWADYMFS
FEPPSGTGYQYYNSLLKTYI
SEQ. ID. NO. 33
ATGAAGTCCCTCTTCGCTCTCAGCCTCTTCGCCGGCCTTTCGGTCGCTCAAAACGCA
GCATGGGCCCAATGCGGTGGAAACGGCTGGACTGGCTCAAAGACCTGCGTCTCTGG
CTACAAGTGCACCGTCGTTAATGAGTGGTACAGCCAGTGTATCCCCGGTACTGCTGA
GGAACCTACTACCACCCTCAAGACTACTACTGGTGGTGGTAGCACACCTACTGGTAC
ACCTGGAAATGGAAAGTTTCTCTGGGTTGGTACTAATGAGGCTGGTGGTGAGTTTGG
TGAGGGCAGTTTGCCTGGAACTTGGGGAAAACACTTTATCTTCCCTGATCCCGCTGC
CGTTGATACCCTCATCTCTCAGGGCTACAACGCCTTCCGCGTTCAACTCCGCATGGA
ACGCACAAACCCTAGCTCCATGACCGGACCCTTTGACACCGCTTACCTCAAGAACCT
CACCACAATCGTGGACCACATCACCGGCAAGGGCGCCAACGTCATTCTCGACCCTC
ACAACTACGGCCGCTACTTTGACAAGATCATCACTTCAACCTCTGACTTCCAGACCT
GGTGGAAGAACTTTGCTACCCAGTTCAAGAGCAACAGCAAAGTCATCTTCGACACT
AACAATGAGTACAACACCATGGATCAGACTCTTGTTCTGAACTTGAACCAGGCTGCT
ATCAACGGTATTCGTGCTGCTGGCGCTACCCAGACTATTTTTGTTGAGGGTAACCAG
TGGTCCGGTGCTTGGTCTTGGCCCGATGTCAACGACAACATGAAGGCTCTCACCGAC
CCTCTTGACAAGATCGTGTACGAAATGCACCAGTACCTCGACTCTGACAGCTCCGGT
ACTTCACCTAACTGTGTCTCCACCACCATTGGAGTCGAGCGCGTCGCGGCTGCTACT
GAGTGGTTGAGGAAGAACAAGAAGATTGGCATGATCGGTGAACTCGCCGGCGGTCC
TAACGACACCTGCAAGACTGCTGTCAAGAATATGCTGGACTATCTTAAGGAGAACTC
TGATGTTTGGAAGGGTGTTACCTGGTGGGCTGCTGGTCCTTGGTGGGCTGACTACAT
GTTCAGCTTTGAGCCTCCCAGCGGTACTGGTTATCAGTACTACAACTCTCTTCTCAAG
ACTTATATCTAA
SEQ. ID. NO. 34
QNAAWAQCGGNGWTGSKTCVSGYKCTVVNEWYSQCIPGTAEEPTTTLKTTTGGGSTPT
GTPGNGKFLWVGTNEAGGEFGEGSLPGTWGKHFIFPDPAAVDTLISQGYNAFRVQLRM
ERTNPSSMTGPFDTAYLKNLTTIVDHITGKGANVILDPHNYGRYFDKIITSTSDFQTWWK
NFATQFKSNSKVIFDTNNEYNTMDQTLVLNLNQAAINGIRAAGATQTIFVEGNQWSGA
WSWPDVNDNMKALTDPLDKIVYEMHQYLDSDSSGTSPNCVSTTIGVERVAAATEWLR
KNKKIGMIGELAGGPNDTCKTAVKNMLDYLKENSDVWKGVTWWAAGPWWADYMFS
FEPPSGTGYQYYNSLLKTYI

What is claimed is:

1. A family 5 glycoside hydrolase variant, the variant encoded by a mutated version of a wild-type parental polynucleotide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or complete sequence identity to a (cDNA) nucleotide sequence of SEQ ID NO: 1 and comprising at least one of the following nucleotide residue changes:
the nucleotides at positions 157 to 159 are TCT;
the nucleotides at positions 175 to 177 are AAG;
the nucleotides at positions 175 to 177 are CAT;
the nucleotides at positions 181 to 183 are CCT;
the nucleotides at positions 190 to 192 are AAT;
the nucleotides at positions 208 to 210 are ATG;
the nucleotides at positions 259 to 261 are CAT;
the nucleotides at positions 259 to 261 are ATG;
the nucleotides at positions 274 to 276 are GCT;
the nucleotides at positions 547 to 549 are TTG;
the nucleotides at positions 550 to 552 are GAG;
the nucleotides at positions 574 to 576 are AAT;
the nucleotides at positions 589 to 591 are GCT;
the nucleotides at positions 595 to 597 are ATG;
the nucleotides at positions 598 to 600 are GTT; and
the nucleotides at positions 898 to 900 are GCG;
wherein the variant has at least one of the following activities: endoglucanase activity, a beta-mannanase activity, an exo-1,3-glucanase activity, an endo-1,6-glucanase activity, a xylanase activity, and an endoglycoceramidase activity; and wherein the activity of the variant is greater than a polypeptide encoded by the wild-type parental polynucleotide having a nucleotide sequence of SEQ ID NO: 1.

2. The family 5 glycoside hydrolase variant of claim 1, wherein family 5 glycoside hydrolase variant has endoglucanase activity.

3. An expression cassette, a vector or a cloning vehicle comprising the mutated version of a wild-type parental polynucleotide sequence SEQ ID NO: 1 of claim 1,
wherein optionally the cloning vehicle comprises a viral vector, a plasmid, a phage, a phagemid, a cosmid, a fosmid, a bacteriophage or an artificial chromosome,
and optionally the viral vector comprises an adenovirus vector, a retroviral vector or an adeno-associated viral vector, and optionally the cloning vehicle comprises a bacterial artificial chromosome (BAC), a plasmid, a bacteriophage P1-derived vector (PAC), a yeast artificial chromosome (YAC), or a mammalian artificial chromosome (MAC).

4. A transformed cell comprising a nucleic acid comprising the mutated version of a wild-type parental polynucleotide sequence SEQ ID NO: 1 of claim 1,
wherein optionally the cell is a bacterial cell, a mammalian cell, a fungal cell, a yeast cell, an insect cell or a plant cell.

5. A transformed cell comprising the expression vector of claim 3.

6. The transformed cell of claim 5, wherein the cell is a bacterial cell, a mammalian cell, a fungal cell, a yeast cell, an insect cell or a plant cell.

7. The transformed cell of claim 6, wherein the bacterial cell is selected from Zymomonas mobilis, Escherichia coli and Klebsiella oxytoca.

8. The transformed cell of claim 6, wherein the yeast cell is selected from Saccharomyces cerevisiae, Saccharomyces uvarum, Kluyveromyces fragilis, Kluyveromyces lactis, Candida pseudotropicalis, and Pachysolen tannophilus.

9. The transformed cell of claim 6, wherein the fungal cell is selected from the genus Aspergillus, Penicillium, Rhizopus, Chrysosporium, Myceliophthora, Trichoderma, Humicola, Acremonium or Fusarium.

10. The transformed cell of claim 9, wherein the fungal cell is of the species Aspergillus niger, Aspergillus oryzae, Trichoderma reesei, Penicillium chrysogenum, Myceliophthora thermophila, or Rhizopus oryzae.

11. The transformed cell of claim 6, wherein the yeast cell is selected from the genus Saccharomyces, Kluyveromyces, Candida, Pichia, Schizosaccharomyces, Hansenula, Klockera, Schwanniomyces or Yarrowia.

12. The transformed cell of claim 11, wherein the yeast cell is of the species S. cerevisiae, S. bulderi, S. barnetti, S. exiguus, S. uvarum, S. diastaticus, K. lactis, K. marxianus or K. fragilis.

13. A mature family 5 glycoside hydrolase variant, the variant having a mutated version of a wild-type parental polypeptide having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or complete sequence identity to an amino acid sequence of SEQ ID NO: 2 and comprising at least one of the following amino acid residue changes:
proline is substituted with serine at position 53;
proline is substituted with histidine at position 59;
proline is substituted with lysine at position 59;
threonine is substituted with proline at position 61;
lysine is substituted with asparagine at position 64;
glycine is substituted with methionine at position 70;
threonine is substituted with histidine at position 87;
threonine is substituted with methionine at position 87;
glycine is substituted with alanine at position 92;
serine is substituted with leucine at position 183;
threonine is substituted with glutamic acid at position 184;
lysine is substituted with asparagine at position 192;
glutamine is substituted with alanine at position 197;
lysine is substituted with methionine at position 199;
serine is substituted with valine at position 200; and
lysine is substituted with alanine at position 300;
wherein the variant has at least one of the following activities: endoglucanase activity, a beta-mannanase activity, an exo-1,3-glucanase activity, an endo-1,6-glucanase activity, a xylanase activity, and an endoglycoceramidase activity; and wherein the activity of the variant is greater than the wild-type parental polypeptide having an amino acid sequence of SEQ ID NO: 2.

14. The variant of claim 13, wherein the variant has endoglucanase activity.

15. A method for hydrolyzing cellulose comprising contacting biomass with the polypeptide of claim 13.

16. The method of claim 15, wherein the biomass is subjected to a pretreatment process prior to being contacted with the polypeptide.

17. The method of claim 16, wherein the pretreatment process comprises the step of heating the biomass to at least 50° Celsius.

18. The method of claim 16, wherein the pretreatment process comprises the step of contacting the biomass with an aqueous solution.

19. The method of claim 18, wherein the aqueous solution has a pH of less than 7.

20. The method of claim 18, wherein the aqueous solution has a pH of more than 7.
